(19) United States
(12) Patent Application Publication
(10) Pub. No.: US 2026/0297494 A1
(43) Pub. Date: Oct. 1, 2026

(54) Experimental Apparatus and Method for Measuring Swimming Features of Algal Cells in Shear Flow

(71) Applicant: CHINA INSTITUTE OF WATER RESOURCES AND HYDROPOWER RESEARCH, BEIJING (CN)

(72) Inventors: Li ZENG, BEIJING (CN); Sheng DING, BEIJING (CN); Bohan WANG, BEIJING (CN); Xinwei HUANG, BEIJING (CN); Qiankun LENG, BEIJING (CN); Kaiyue ZHONG, BEIJING (CN); Xiaoli CHEN, BEIJING (CN); Rui HAN, BEIJING (CN); Haiwen ZHANG, BEIJING (CN); Yijun ZHAO, BEIJING (CN); Yan LIU, BEIJING (CN); Zanqiang LIU, BEIJING (CN); Honghua LIANG, BEIJING (CN); Qiang ZHANG, BEIJING (CN)

(73) Assignee: CHINA INSTITUTE OF WATER RESOURCES AND HYDROPOWER RESEARCH, BEIJING (CN)

(21) Appl. No.: 19/372,289

(22) Filed: Oct. 29, 2025

(30) Foreign Application Priority Data
Mar. 27, 2025 (CN) 202510372305.3

(51) Int. Cl. C12M 1/34; C12M 1/00; C12M 1/42

(52) U.S. Cl. CPC C12M 41/46; C12M 29/00; C12M 35/04; C12M 41/40; C12M 41/44

(57) ABSTRACT

The present invention discloses an experimental apparatus and a method for measuring swimming features of algal cells in a shear flow, wherein the experimental apparatus comprises an air pressure source, a pressure pump, a liquid storage bottle, a flow meter, a microchannel and a waste liquid bottle which are connected in sequence; the method comprises: determining an average flow rate according to the experimental conditions to produce a stable shear flow; capturing an image sequence of the movement track of algal cells; and observing the movement track of algae cells to obtain the distribution of an algae cell migration velocity V, obtain the distribution of an algae cell flow velocity V_{flow} by combining the analytic method, and then obtain the swimming features of the algae cells in the shear flow, comprising a swimming velocity Vₛ and an angular velocity ω of the algae cells. The present invention achieves high-precision measurement of algal cell movement in shear flow, combines experimental methods with analytical methods, and overcomes the shortcomings of previous studies that cannot obtain the swimming features of algal cells, such as swimming velocity and angular velocity, and provides technical support for the development of microbial hydraulics on a more detailed micro-scale.

### CROSS-REFERENCE TO RELATED APPLICATION

This application claims the priority benefit to Chinese application No. 202510372305.3 filed on Mar. 27, 2025, the entirety of which is hereby incorporated by reference and made a part of this specification.

### TECHNICAL FIELD

The present invention belongs to the technical field of environmental hydraulics, and more particularly, to an experimental apparatus and a method for measuring swimming features of algae cells in shear flow.

### BACKGROUND ART

At present, the problems of water environment pollution and water ecological damage caused by water bloom have become increasingly prominent. Therefore, it is of great significance to clarify the swimming features of algae cells for understanding the laws of algae migration, aggregation and distribution, and then predicting the occurrence and development process of water bloom. Flagellate bloom is a widespread phenomenon in river-type reservoirs and gulfs. Flagellate bloom can be driven by its own flagella, which is not only related to its own dynamic characteristics, but also significantly affected by external flow conditions.

Shear flow exists widely in natural and artificial environment, from ocean turbulence to river flow, and then to the liquid circulation in bioreactor, all of which contain significant shear effect. Therefore, the precise observation of algae cell movement in shear flow is very important to reveal the interaction between water flow and microalgae movement. The swimming features of algae cells mainly include swimming velocity, angular velocity and so on. At present, the observation of algae cell swimming features by micro-scale experiments in shear flow environment is still immature, and there is no corresponding research results in China. Therefore, how to measure the swimming features of algae cells in shear flow is an urgent technical problem to be solved.

### SUMMARY

It is an object of the present invention to provide an experimental apparatus and a method for measuring swimming features of algae cells in shear flow, so as to solve the above-mentioned technical problem.

In order to achieve the above object, the present invention provides the technical solutions below.

The present invention discloses an experimental apparatus for measuring swimming features of algal cells in a shear flow, the experimental apparatus comprises an air pressure source, a pressure pump, a liquid storage bottle, a flow meter, a microchannel and a waste liquid bottle which are connected in sequence; the air pressure source is connected to the pressure pump, and the pressure pump is connected to the liquid storage bottle via a connecting tube; the liquid storage bottle is connected to the flow meter, the flow meter is connected to an inlet of the microchannel, and an outlet of the microchannel is connected to the waste liquid bottle, via a conduit; the apparatus further comprises a fluorescence microscope and a high-speed camera which are connected; the high-speed camera is installed below an objective table of the fluorescence microscope; the microchannel is arranged on an objective table of a fluorescence microscope; the air pressure source is configured for providing a base pressure for the pressure pump; the pressure pump is configured for outputting a set constant pressure so as to create an internal-external pressure difference in the liquid storage bottle; the liquid storage bottle is a closed type with an algae cell solution therein; the algae cell solution is conveyed to the microchannel via a conduit to form a shear flow under the action of the internal and external pressure difference; the flow meter is configured for monitoring a flow rate pumped by the pressure pump in real time; the microchannel is configured for forming a shear flow; the waste liquid bottle is configured for collecting the waste solution of algae cells flowing out of the outlet of the microchannel; the fluorescence microscope is configured for manufacturing a fluorescence field environment, inducing the algae cells to generate a characteristic fluorescence signal, synchronously achieving microscopic image amplification and filtering out other stray light; and the high-speed camera is configured for shooting an image sequence of the algae cells in the shear flow.

Further, the inlet, the outlet and the conduit of the microchannel are all connected by a Luer fitting.

Further, the microchannel is a rectangular parallelepiped having a length of 5 cm, a width of 600 μm, and a height of 150 μm; and the microchannels are cast from PDMS materials or etched from silicon materials.

The invention also discloses a method for measuring swimming features of algal cells in shear flow, the method comprising the steps of:
step 1, preparing a solution of algae cells with a certain concentration, connecting the experimental apparatus and checking the air tightness of the experimental apparatus;
step 2, adjusting the position of the microchannel, so that the center of a picture shot by the high-speed camera corresponds to a center position of the microchannel, and front and rear wall surfaces of the microchannel are parallel to a long frame of the picture shot;
step 3, determining an average flow rate according to the experimental working condition, setting the pressure in the liquid storage bottle by a pressure pump, and continuously adjusting the pressure size according to the flow rate monitored by the flow meter in real time, until the measured flow rate remains stable and consistent with the required average flow rate, so as to produce a stable shear flow;
step 4, firstly, capturing an image in a bright field environment by the high-speed camera so as to calibrate the front and rear wall surfaces of the microchannel, namely, respectively corresponding to upper and lower boundaries of the captured image; then, turning off the light source, and capturing the image sequence in the environment of fluorescence field, wherein the recording duration of each group of experiments is five minutes, and the experiments are repeated several times until the effective movement tracks of algal cells reach tens of thousands;
step 5, processing the captured image sequence, setting a diameter and a brightness threshold to identify the algae cells, extracting coordinate data of the movement track of algae cells, and eliminating tracks outside the observation area according to the upper and lower boundaries of the calibrated captured image, and only keeping tracks inside the observation area;
step 6, observing the movement track of algae cells to obtain the distribution of an algae cell migration velocity V, obtain the distribution of an algae cell flow velocity V_{flow} by combining the analytic method, and then obtain the swimming features of the algae cells in the shear flow, comprising a swimming velocity Vₛ and an angular velocity ω of the algae cells; it specifically comprises the steps of:
step 61, calculating a migration velocity component of the algae cells: according to the position coordinates of the movement track of algae cells observed in the experiment, calculating other points on the track by the central difference format except for a first point using the forward difference and a last point using the backward difference to obtain migration velocity components Vₓ, V_{y} of the algae cell at different positions of each track;
   the first point:

$$\begin{matrix}{V}_{x 1} = \frac{{x}_{2} - {x}_{1}}{Δ t} & {V}_{y 1} = \frac{{y}_{2} - {y}_{1}}{Δ t}\end{matrix} \qquad ( 1 )$$

the last point:

$$\begin{matrix}{V}_{x end} = \frac{{x}_{end} - {x}_{end - 1}}{Δ t} & {V}_{y end} = \frac{{y}_{end} - {y}_{end - 1}}{Δ t}\end{matrix} \qquad ( 2 )$$

$$\begin{matrix}{V}_{x i} = \frac{{x}_{i + 1} - {x}_{i - 1}}{2 Δ t} & {V}_{y i} = \frac{{y}_{i + 1} - {y}_{i - 1}}{2 Δ t}\end{matrix} \qquad ( 3 )$$

in the formula, x, y are the coordinates of the algae cell, and their subscripts indicate the coordinates, μm, of different points; Vₓ and V_{y} are the migration velocity components respectively, and their subscripts indicate the migration velocity components, μm/s, of different points; Δt is the time interval, s, of the adjacent points on the track;
step 62, calculating a flow velocity distribution of the algae cells, wherein, in a shear flow environment, the component of the flow velocity at different positions along the width direction of the microchannel is 0 in the y direction, and the component V_{flow_x} in the x direction is obtained by an analytical method; the calculation formula is as follows:

$${V}_{flow \_ x} = \frac{16 {a}^{2}}{{μπ}^{3}} ( - \frac{d \hat{p}}{dx} ) {\sum }_{i = 1 , 3 , 5 …}^{∞} {( - 1 )}^{( i - 1 ) / 2} \times [ 1 - \frac{1}{cosh ( i π b / 2 a )} ] \times \frac{cos ( i π y / 2 a )}{{i}^{3}} \qquad ( 4 )$$

$$Q = \frac{4 {ba}^{3}}{3 μ} ( - \frac{d \hat{p}}{dx} ) [ 1 - \frac{192 a}{{π}^{5} b} {\sum }_{i = 1 , 3 , 5 …}^{∞} \frac{tanh ( i π b / 2 a )}{{i}^{5}} ] \qquad ( 5 )$$

in the Formula, a and b are respectively half of the width and height, μm, of the microchannel; \hat{p} is the pressure, Kg μm⁻¹s⁻²; μ is the dynamic viscosity, Kg μm⁻¹s⁻¹, of liquid; i is an index variable for the summation of the series; Q is an average flow rate, μm³/s;
step 63, calculating the swimming velocity of the algae cell itself and a component thereof: dividing the migration velocity V of algae cells in shear flow into two parts: the self-swimming velocity Vₛ, i. e., the swimming velocity of algae cells actively generated by flagellar driving, and the flow velocity V_{flow}, i. e., the velocity of water flow at the position of algae cells; the migration velocity Vis a superposition of the self-swimming velocity Vₛ and the flow velocity V_{flow}, i. e.,

$$V = {V}_{s} + {V}_{flow} \qquad ( 6 )$$

in the Formula, the units of V, Vₛ and V_{flow} are μm/s;
calculating the swimming velocity Vₛ of algae cell and its components V_{s_x}, V_{s_y} according to the migration velocity of algae cells and the flow velocity at the position of algae cells, with the calculation formula as follows:

$${V}_{s \_ x} = {V}_{x} - {V}_{flow \_ x} \qquad ( 7 )$$

$${V}_{s \_ y} = {V}_{y} \qquad ( 8 )$$

$${V}_{s} = \sqrt{{V}_{s \_ x}^{2} + {V}_{s \_ y}^{2}} \qquad ( 9 )$$

step 64, calculating the angular velocity of the algae cells: according to the component of the swimming velocity of algae cell, calculating its movement direction θ, with the calculation formula as follows:

$$θ = arctan \frac{{V}_{s \_ y}}{{V}_{s \_ x}} \qquad ( 10 )$$

calculating the movement direction change of the algal cell according to the movement direction of each track of the algal cell at different moments so as to obtain the angular velocity w of the algal cell; in the calculation method, calculating other points on the track by the central difference format except for a first point using the forward difference and a last point using the backward difference;
the first point:

$${ω}_{1} = \frac{{θ}_{2} - {θ}_{1}}{Δ t} \qquad ( 11 )$$

the last point:

$${ω}_{end} = \frac{{θ}_{end} - {θ}_{end - 1}}{Δ t} \qquad ( 12 )$$

the other points:

$${ω}_{i} = \frac{{θ}_{i + 1} - {θ}_{i - 1}}{2 Δ t} \qquad ( 13 )$$

in the formula, θ is the direction, rad, of algal cell movement; Ω is the angular velocity, rad/s, of the algae cell; the subscripts indicate the movement direction and angular velocity of different points; and
step 65, measuring swimming features of algal cells: according to the track analysis of a large number of algal cells, counting a swimming velocity and an angular velocity of the algal cells at different positions along the width direction of the microchannel, and average processing to obtain the distribution of the swimming velocity and angular velocity of the algal cells themselves along the width direction of the microchannel, that is to say, achieving the measurement of the swimming features of the algal cells at different shear rates.

Further, the concentration of the algal cell solution prepared in the step 1 is 1.2×10⁵ cells/ml.

Furthermore, in the step 4, the criteria for determining the effective movement track of algal cells is as follows:
1) the time of the movement track of algae cells is longer than 10 s; and
2) the recorded track does not include a track that is stationary and a track that adheres to impurities in certain areas.

Further, in the step 5, the diameter threshold is set according to the type of algae cells, and the sizes of different types of algae cells are different; and the brightness threshold is set according to wavelength band, intensity and exposure time of the laser. Further, the plurality of tracks of the algal cells in the step 65 is at least ten thousand or more tracks of the algal cells.

The beneficial effects of the present invention are that the advantageous effects of the present invention are embodied in the following aspects.

1. In the present invention, the shear flow is produced by the pressure pump which drives a stable flow of algae solution based on the internal and external pressure difference of a closed liquid storage bottle by outputting a constant pressure, so as to achieve pulse-free fluid delivery. Compared with the commonly used syringe pump, the pressure pump has higher accuracy and better stability, so as to ensure the reliability of shear flow environment in the experiment. In addition, the pressure pump is also connected with a high-sensitivity flow meter with or without contact, so that the flow changes can be monitored in real time during the experiment, and the output pressure of the pressure pump can be adjusted appropriately.

2. In present invention the distribution of the migration velocity V of the algae cell is obtained by the observed movement track of the algae cell, and the distribution of the flow velocity V_{flow} of the algae cell is obtained by combining with the analytical method. The swimming velocity Vₛ and the angular velocity ω of the algae cell itself are then calculated, so as to realize the measurement of the swimming features of the algae cell.

3. The present invention achieves high-precision measurement of algal cell movement in shear flow, combines experimental methods with analytical methods, and overcomes the shortcomings of previous studies that cannot obtain the swimming features of algal cells, such as swimming velocity and angular velocity, and provides technical support for the development of microbial hydraulics on a more detailed micro-scale.

Hereinafter, the present invention will be described in further detail with reference to the accompanying drawings and detailed description.

### BRIEF DESCRIPTION OF THE DRAWINGS

FIG. 1 is a structurally schematic diagram of an experimental apparatus;

FIG. 2 is a structurally schematic diagram of a microchannel;

FIG. 3 is a chart of a partial track of the movement of algae cells observed in Example 1;

FIG. 4 is a distribution diagram showing a component Vₓ of a migration velocity of algae cells in Example 1;

FIG. 5 is a distribution diagram showing V_{s_x}, a component of a swimming velocity of the algae cell itself in Example 1;

FIG. 6 is a distribution diagram showing V_{s_y}, a component of the swimming velocity of the algae cell itself in Example 1; and

FIG. 7 is a distribution diagram showing angular velocity ω of the algae cells in Example 1.

In the drawings: 1, pressure pump; 2, air pressure source; 3, liquid storage bottle; 4, flow meter; 5, microchannel; 6, Luer fitting; 7, conduit; 8, waste liquid bottle.

### DETAILED DESCRIPTION OF THE INVENTION

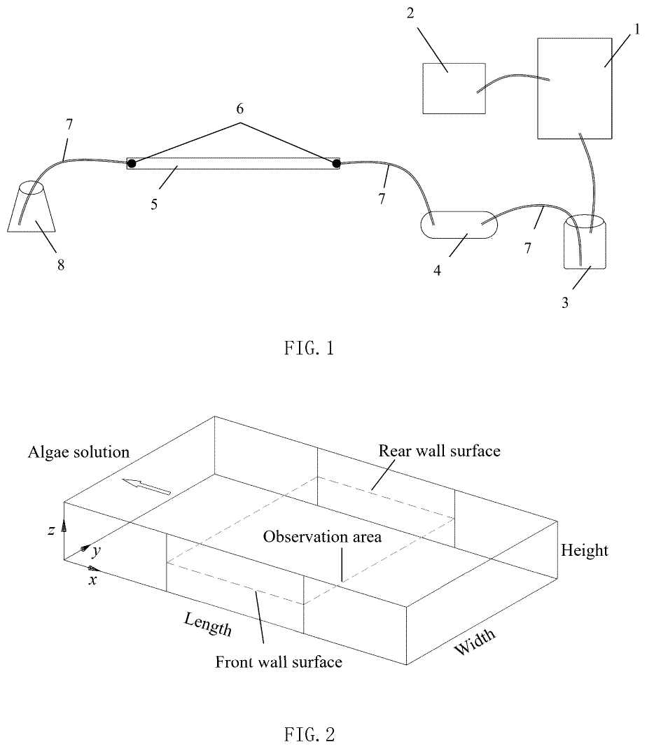

The present invention discloses an experimental apparatus and a method for measuring the swimming features of algal cells in the shear flow. As shown in FIG. 1, the experimental apparatus comprises an air pressure source 2, a pressure pump 1, a liquid storage bottle 3, a flow meter 4, a microchannel 5 and a waste liquid bottle 8 which are connected in sequence. The air pressure source and the pressure pump, and the pressure pump and the liquid storage bottle are all connected via a connecting tube. The liquid storage bottle and the flow meter, the flow meter and the inlet of the microchannel, and the outlet of the microchannel and the waste liquid bottle are all connected via a conduit 7. The inlet, the outlet and the conduit of the microchannel are all connected via a Luer fitting 6. The apparatus further comprises a fluorescence microscope and a high-speed camera connected thereto, the high-speed camera being mounted below the stage of the fluorescence microscope, and the microchannel being arranged on the objective table of the fluorescence microscope. The air pressure source provides the base pressure for the pressure pump. The pressure pump outputs a set constant pressure to make the internal and external pressure difference in the liquid storage bottle. The liquid storage bottle is a closed type with an algae cell solution therein. Under the action of an internal and external pressure difference, the algae cell solution is conveyed to a microchannel via a conduit to form a shear flow. The flow meter is configured for monitoring of the flow pumped by the pressure pump in real time. The microchannel is an area where algae solution flows, and is used in combination with a pressure pump to produce a stable and accurate shear flow. The waste liquid bottle is configured for collecting the algae cell waste solution flowing out of the outlet of the microchannel. The fluorescence microscope is configured for manufacturing a fluorescence field environment, inducing the algae cells to generate a characteristic fluorescence signal, synchronously achieving microscopic image amplification and filtering out other stray light. The high-speed camera is configured for shooting an image sequence of the algae cells in the shear flow.

As shown in FIG. 2, the microchannel is a rectangular parallelepiped having a length of 5 cm, a width of 600 μm, and a height of 150 μm; and the microchannels are cast from PDMS materials or etched from silicon materials.

The invention also discloses a method for measuring swimming features of algal cells in shear flow, the method comprising the steps below.

Step 1, a solution of algae cells with a certain concentration is prepared, the experimental apparatus is connected and the air tightness of the experimental apparatus is checked.

Step 2, the position of the microchannel is adjusted, so that the center of a picture shot by the high-speed camera corresponds to a center position of the microchannel; the observation area is in the middle of the inlet and the outlet of the channel; and front and rear wall surfaces of the microchannel are parallel to a long frame of the picture shot.

Step 3, an average flow rate (or shear rate) is determined according to the experimental working condition, the pressure in the liquid storage bottle is set by a pressure pump, and the pressure size is continuously adjusted according to the flow rate monitored by the flow meter in real time, until the measured flow rate remains stable and consistent with the required average flow rate, so as to produce a stable shear flow; and

step 4, firstly, capturing an image in a bright field environment by the high-speed camera so as to calibrate the front and rear wall surfaces of the microchannel, namely, respectively corresponding to upper and lower boundaries of the captured image; then, turning off the light source, and capturing the image sequence in the environment of fluorescence field, wherein the recording duration of each group of experiments is five minutes, and the experiments are repeated several times until the effective movement tracks of algal cells reach tens of thousands. Typically, a frame rate of 100 frames is taken, i. e., 30000 images are taken per set of experiments.

Specifically, an effective algal cell movement track refers to a long-time algal cell movement track in the observation area, which can reflect the normal movement of algal cells in the shear flow. The following conditions are required be met.

1) The time to record the movement track of algae cells is longer than 10 s (the time can be adjusted according to the specific experimental conditions).

2) The tracks recorded do not include the tracks of algae cells with weak movement ability (such as static tracks) as well as the tracks of algae cells that are misidentified (such as the tracks of impurities adhering to certain areas).

Step 5, processing the recorded image sequence by using the trackmate open source program, setting an appropriate diameter and a brightness threshold to identify the algae cells, extracting coordinate data of the movement track of algae cells, and eliminating tracks outside the observation area according to the upper and lower boundaries of the calibrated captured image, and only keeping tracks inside the observation area;

The diameter threshold is set according to the type of algae cell, and the size of different types of algae cells is different. The brightness threshold is set according to the wavelength band, intensity and exposure time of the laser.

Step 6, observing the movement track of algae cells to obtain the distribution of an algae cell migration velocity V, obtain the distribution of an algae cell flow velocity V_{flow} by combining the analytic method, and then obtain the swimming features of the algae cells in the shear flow, comprising a swimming velocity Vₛ and an angular velocity ω of the algae cells; it specifically comprises the steps of:

step 61, calculating a migration velocity component of the algae cells: according to the position coordinates of the movement track of algae cells observed in the experiment, calculating other points on the track by the central difference format except for a first point using the forward difference and a last point using the backward difference to obtain migration velocity components Vₓ, V_{y} of the algae cell at different positions of each track;
the first point:

$$\begin{matrix}{V}_{x 1} = \frac{{x}_{2} - {x}_{1}}{Δ t} & {V}_{y 1} = \frac{{y}_{2} - {y}_{1}}{Δ t}\end{matrix} \qquad ( 1 )$$

the last point:

$$\begin{matrix}{V}_{x end} = \frac{{x}_{end} - {x}_{end - 1}}{Δ t} & {V}_{y end} = \frac{{y}_{end} - {y}_{end - 1}}{Δ t}\end{matrix} \qquad ( 2 )$$

the other points:

$$\begin{matrix}{V}_{x i} = \frac{{x}_{i + 1} - {x}_{i - 1}}{2 Δ t} & {V}_{y i} = \frac{{y}_{i + 1} - {y}_{i - 1}}{2 Δ t}\end{matrix} \qquad ( 3 )$$

in the formula, x, y are the coordinates of the algae cell, and their subscripts indicate the coordinates, μm, of different points; Vₓ and V_{y} are the migration velocity components respectively, and their subscripts indicate the migration velocity components, μm/s, of different points; Δt is the time interval, s, of the adjacent points on the track;
step 62, calculating a flow velocity distribution of the algae cells, wherein, in a shear flow environment, the component of the flow velocity at different positions along the width direction of the microchannel is 0 in the y direction, and the component V_{flow_x} in the x direction is obtained by an analytical method; the calculation formula is as follows:

$${V}_{flow \_ x} = \frac{16 {a}^{2}}{{μπ}^{3}} ( - \frac{d \hat{p}}{dx} ) {\sum }_{i = 1 , 3 , 5 …}^{∞} {( - 1 )}^{( i - 1 ) / 2} \times [ 1 - \frac{1}{cosh ( i π b / 2 a )} ] \times \frac{cos ( i π y / 2 a )}{{i}^{3}} \qquad ( 4 )$$

$$Q = \frac{4 {ba}^{3}}{3 μ} ( - \frac{d \hat{p}}{dx} ) [ 1 - \frac{192 a}{{π}^{5} b} {\sum }_{i = 1 , 3 , 5 …}^{∞} \frac{tanh ( i π b / 2 a )}{{i}^{5}} ] \qquad ( 5 )$$

in the Formula, a and b are respectively half of the width and height, μm, of the microchannel; p\hat{ } is the pressure, Kg μm⁻¹s⁻²; μ is the dynamic viscosity, Kg μm⁻¹s⁻¹, of liquid; i is an index variable for the summation of the series; Q is an average flow rate, μm³/s;
step 63, calculating the swimming velocity of the algae cell itself and a component thereof: dividing the migration velocity V of algae cells in shear flow into two parts: the self-swimming velocity Vₛ, i. e., the swimming velocity of algae cells actively generated by flagellar driving, and the flow velocity V_{flow}, i. e., the velocity of water flow at the position of algae cells; the migration velocity Vis a superposition of the self-swimming velocity Vₛ and the flow velocity V_{flow}, i. e.,

$$V = {V}_{s} + {V}_{flow} \qquad ( 6 )$$

in the Formula, the units of V, Vₛ and V_{flow} are μm/s;
calculating the swimming velocity Vₛ of algae cell and its components V_{s_x}, V_{s_y} according to the migration velocity of algae cells and the flow velocity at the position of algae cells, with the calculation formula as follows:

$${V}_{s \_ x} = {V}_{x} - {V}_{flow \_ x} \qquad ( 7 )$$

$${V}_{s \_ y} = {V}_{y} \qquad ( 8 )$$

$${V}_{s} = \sqrt{{V}_{s \_ x}^{2} + {V}_{s \_ y}^{2}} \qquad ( 9 )$$

step 64, calculating the angular velocity of the algae cells: according to the component of the swimming velocity of algae cell, calculating its movement direction θ, with the calculation formula as follows:

$$θ = arctan \frac{{V}_{s \_ y}}{{V}_{s \_ x}} \qquad ( 10 )$$

calculating the movement direction change of the algal cell according to the movement direction of each track of the algal cell at different moments so as to obtain the angular velocity ω of the algal cell; with the calculation method consistent with the calculation speed, calculating other points on the track by the central difference format except for a first point using the forward difference and a last point using the backward difference,
the first point:

$${ω}_{1} = \frac{{θ}_{2} - {θ}_{1}}{Δ t} \qquad ( 11 )$$

the last point:

$${ω}_{end} = \frac{{θ}_{end} - {θ}_{end - 1}}{Δ t} \qquad ( 12 )$$

the other points:

$${ω}_{i} = \frac{{θ}_{i + 1} - {θ}_{i - 1}}{2 Δ t} \qquad ( 13 )$$

in the formula, θ is the direction, rad, of algal cell movement; Ω is the angular velocity, rad/s, of the algae cell; the subscripts indicate the movement direction and angular velocity of different points; and
step 65, measuring swimming features of algal cells: due to the parabolic distribution of the water flow velocity along the width direction, the shear rate varies at different locations along the width direction. According to the analysis of the movement track of a large number of algae cells (at least 10,000 or more), the swimming velocity and angular velocity of algae cells at different positions along the width direction of the microchannel are counted, and the distribution of the swimming velocity and angular velocity of algae cells along the width direction of the microchannel is obtained by average processing, i. e., the swimming features of algae cells under different shear rates are measured.

### Example 1

The present embodiment is an application example of the above-mentioned method. The concentration of the algal cell solution prepared in this example is 1.2×10⁵ cells/ml, and the algal cell type used is Heterosigma akashiwo, with a cell diameter of about 10 μm. In this example, the swimming features of algae cells are measured using a shear flow with an average flow rate of 9×10⁶ μm³.

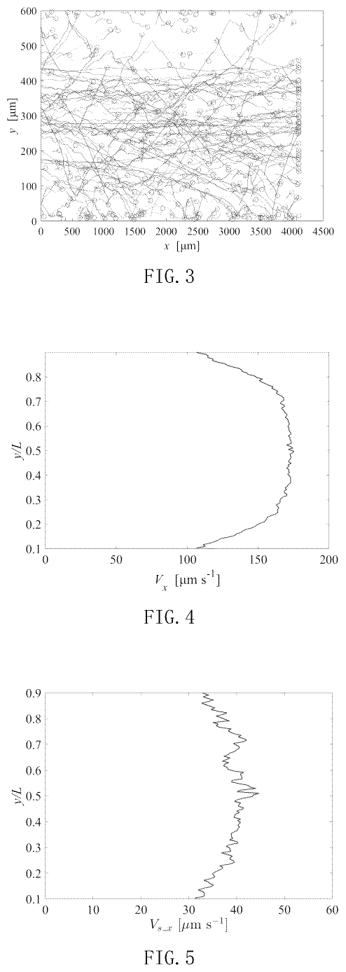

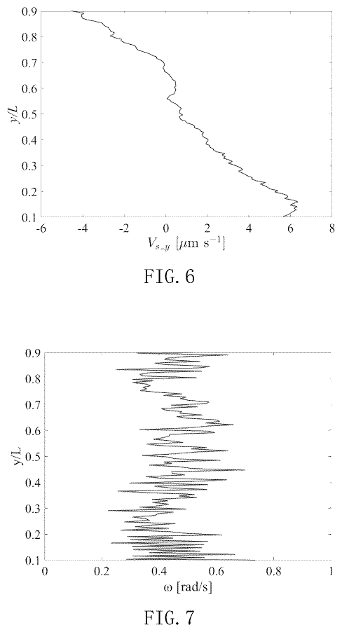

The movement track of algae cells is observed by using the above experimental apparatus, and as shown in FIG. 3, it is a randomly selected partial track, and the circle in the figure is the initial position of the track. The diameter threshold is set at 12 pixel, and the brightness threshold is set at 20. FIG. 4 is a graph showing an experimentally measured component Vₓ of the migration velocity of algal cells. FIG. 5 and FIG. 6 are graphs showing the components V_{s_x} and V_{s_y} of the swimming velocity of the algae cells obtained by the above-described method, respectively. FIG. 7 is a distribution diagram showing angular velocity ω of algae cells obtained by the above-described method.

Finally, it should be stated that the above descriptions are merely intended to illustrate the technical solutions of the present invention rather than to limit them. Although the present invention has been described in detail with reference to preferred embodiments, those of ordinary skill in the art should understand that modifications or equivalent replacements to the technical solutions of the present invention may be made without departing from the spirit and scope thereof.

1. A method for measuring swimming features of algal cells in shear flow, wherein the method is based on an experimental apparatus; the experimental apparatus comprises an air pressure source, a pressure pump, a liquid storage bottle, a flow meter, a microchannel and a waste liquid bottle which are connected in sequence; the air pressure source is connected to the pressure pump, and the pressure pump is connected to the liquid storage bottle via a connecting tube; the liquid storage bottle is connected to the flow meter, the flow meter is connected to an inlet of the microchannel, and an outlet of the microchannel is connected to the waste liquid bottle, via a conduit; the apparatus further comprises a fluorescence microscope and a high-speed camera which are connected; the high-speed camera is installed below an objective table of the fluorescence microscope; the microchannel is arranged on an objective table of a fluorescence microscope; the air pressure source is configured for providing a base pressure for the pressure pump; the pressure pump is configured for outputting a set constant pressure so as to create an internal-external pressure difference in the liquid storage bottle; the liquid storage bottle is a closed type with an algae cell solution therein; the algae cell solution is conveyed to the microchannel via a conduit to form a shear flow under the action of the internal and external pressure difference; the flow meter is configured for monitoring a flow rate pumped by the pressure pump in real time; the microchannel is configured for forming a shear flow; the waste liquid bottle is configured for collecting the waste solution of algae cells flowing out of the outlet of the microchannel; the fluorescence microscope is configured for manufacturing a fluorescence field environment, inducing the algae cells to generate a characteristic fluorescence signal, synchronously achieving microscopic image amplification and filtering out other stray light; the high-speed camera is configured for shooting an image sequence of the algae cells in the shear flow;
wherein the method comprises the steps of:
step 1, preparing a solution of algae cells with a certain concentration, connecting the experimental apparatus and checking the air tightness of the experimental apparatus;
step 2, adjusting the position of the microchannel, so that the center of a picture shot by the high-speed camera corresponds to a center position of the microchannel, and front and rear wall surfaces of the microchannel are parallel to a long frame of the picture shot;
step 3, determining an average flow rate according to the experimental working condition, setting the pressure in the liquid storage bottle by a pressure pump, and continuously adjusting the pressure size according to the flow rate monitored by the flow meter in real time, until the measured flow rate remains stable and consistent with the required average flow rate, so as to produce a stable shear flow;
step 4, firstly, capturing an image in a bright field environment by the high-speed camera so as to calibrate the front and rear wall surfaces of the microchannel, namely, respectively corresponding to upper and lower boundaries of the captured image; then, turning off the light source, and capturing the image sequence in the environment of fluorescence field, wherein the recording duration of each group of experiments is five minutes, and the experiments are repeated several times until the effective movement tracks of algal cells reach tens of thousands;
step 5, processing the captured image sequence, setting a diameter and a brightness threshold to identify the algae cells, extracting coordinate data of the movement track of algae cells, and eliminating tracks outside the observation area according to the upper and lower boundaries of the calibrated captured image, and only keeping tracks inside the observation area;
step 6, observing the movement track of algae cells to obtain the distribution of an algae cell migration velocity V, obtain the distribution of an algae cell flow velocity V_{flow} by combining the analytic method, and then obtain the swimming features of the algae cells in the shear flow, comprising a swimming velocity Vₛ and an angular velocity ω of the algae cells; it specifically comprises the steps of:
step 61, calculating a migration velocity component of the algae cells: according to the position coordinates of the movement track of algae cells observed in the experiment, calculating other points on the track by the central difference format except for a first point using the forward difference and a last point using the backward difference to obtain migration velocity components Vₓ, V_{y} of the algae cell at different positions of each track;
the first point:
$$\begin{matrix}{V}_{x 1} = \frac{{x}_{2} - {x}_{1}}{Δ t} & {V}_{y 1} = \frac{{y}_{2} - {y}_{1}}{Δ t}\end{matrix} \qquad ( 1 )$$
the last point:
$$\begin{matrix}{V}_{x end} = \frac{{x}_{end} - {x}_{end - 1}}{Δ t} & {V}_{y end} = \frac{{y}_{end} - {y}_{end - 1}}{Δ t}\end{matrix} \qquad ( 2 )$$
the other points:
$$\begin{matrix}{V}_{x i} = \frac{{x}_{i + 1} - {x}_{i - 1}}{2 Δ t} & {V}_{y i} = \frac{{y}_{i + 1} - {y}_{i - 1}}{2 Δ t}\end{matrix} \qquad ( 3 )$$
in the formula, x, y are the coordinates of the algae cell, and their subscripts indicate the coordinates, μm, of different points; Vₓ and V_{y} are the migration velocity components respectively, and their subscripts indicate the migration velocity components, μm/s, of different points; Δt is the time interval, s, of the adjacent points on the track;
step 62, calculating a flow velocity distribution of the algae cells, wherein, in a shear flow environment, the component of the flow velocity at different positions along the width direction of the microchannel is 0 in the y direction, and the component V_{flow_x} in the x direction is obtained by an analytical method; the calculation formula is as follows:
$${V}_{flow \_ x} = \frac{16 {a}^{2}}{{μπ}^{3}} ( - \frac{d \hat{p}}{dx} ) {\sum }_{i = 1 , 3 , 5 …}^{∞} {( - 1 )}^{( i - 1 ) / 2} \times [ 1 - \frac{1}{cosh ( i π b / 2 a )} ] \times \frac{cos ( i π y / 2 a )}{{i}^{3}} \qquad ( 4 )$$
$$Q = \frac{4 {ba}^{3}}{3 μ} ( - \frac{d \hat{p}}{dx} ) [ 1 - \frac{192 a}{{π}^{5} b} {\sum }_{i = 1 , 3 , 5 …}^{∞} \frac{tanh ( i π b / 2 a )}{{i}^{5}} ] \qquad ( 5 )$$
in the Formula, a and b are respectively half of the width and height, μm, of the microchannel; \hat{p} is the pressure, Kg μm⁻¹s⁻²; μ is the dynamic viscosity, Kg μm⁻¹s⁻¹, of liquid; i is an index variable for the summation of the series; Q is an average flow rate, μm³/s;
step 63, calculating the swimming velocity of the algae cell itself and a component thereof: dividing the migration velocity V of algae cells in shear flow into two parts: the self-swimming velocity Vₛ, i. e., the swimming velocity of algae cells actively generated by flagellar driving, and the flow velocity V_{flow}, i. e., the velocity of water flow at the position of algae cells; the migration velocity Vis a superposition of the self-swimming velocity Vₛ and the flow velocity V_{flow}, i. e.,
$$V = {V}_{s} + {V}_{flow} \qquad ( 6 )$$
in the Formula, the units of V, Vₛ and V_{flow} are μm/s;
calculating the swimming velocity Vₛ of algae cell and its components V_{s_x}, V_{s_y} according to the migration velocity of algae cells and the flow velocity at the position of algae cells, with the calculation formula as follows:
$${V}_{s \_ x} = {V}_{x} - {V}_{flow \_ x} \qquad ( 7 )$$
$${V}_{s \_ y} = {V}_{y} \qquad ( 8 )$$
$${V}_{s} = \sqrt{{V}_{s \_ x}^{2} + {V}_{s \_ y}^{2}} \qquad ( 9 )$$
step 64, calculating the angular velocity of the algae cells: according to the component of the swimming velocity of algae cell, calculating its movement direction θ, with the calculation formula as follows:
$$θ = arctan \frac{{V}_{s \_ y}}{{V}_{s \_ x}} \qquad ( 10 )$$
calculating the movement direction change of the algal cell according to the movement direction of each track of the algal cell at different moments so as to obtain the angular velocity ω of the algal cell; in the calculation method, calculating other points on the track by the central difference format except for a first point using the forward difference and a last point using the backward difference;
the first point:
$${ω}_{1} = \frac{{θ}_{2} - {θ}_{1}}{Δ t} \qquad ( 11 )$$
the last point:
$${ω}_{end} = \frac{{θ}_{end} - {θ}_{end - 1}}{Δ t} \qquad ( 12 )$$
the other points:
$${ω}_{i} = \frac{{θ}_{i + 1} - {θ}_{i - 1}}{2 Δ t} \qquad ( 13 )$$
in the formula, θ is the direction, rad, of algal cell movement; Ω is the angular velocity, rad/s, of the algae cell; the subscripts indicate the movement direction and angular velocity of different points; and
step 65, measuring swimming features of algal cells: according to the track analysis of a large number of algal cells, counting a swimming velocity and an angular velocity of the algal cells at different positions along the width direction of the microchannel, and average processing to obtain the distribution of the swimming velocity and angular velocity of the algal cells themselves along the width direction of the microchannel, that is to say, achieving the measurement of the swimming features of the algal cells at different shear rates.

2. The method for measuring swimming features of algal cells in shear flow according to claim 1, wherein the inlet, the outlet and the conduit of the microchannel are all connected by a Luer fitting.

3. The method for measuring swimming features of algal cells in shear flow according to claim 1, wherein the microchannel is a rectangular parallelepiped having a length of 5 cm, a width of 600 μm, and a height of 150 μm; and the microchannels are cast from PDMS materials or etched from silicon materials.

4. The method for measuring swimming features of algal cells in shear flow according to claim 1, wherein the concentration of the algal cell solution prepared in the step 1 is 1.2×10⁵ cells/ml.

5. The method for measuring swimming features of algal cells in shear flow according to claim 1, wherein, in the step 4, the criteria for determining the effective movement track of algae cells are as follows:
1) the time of the movement track of algae cells is longer than 10 s; and
2) the recorded track does not include a track that is stationary and a track that adheres to impurities in certain areas.

6. The method for measuring swimming features of algal cells in shear flow according to claim 1, wherein, in the step 5, the diameter threshold is set according to the type of algae cells, and the sizes of different types of algae cells are different; and the brightness threshold is set according to wavelength band, intensity and exposure time of the laser.

7. The method for measuring swimming features of algal cells in shear flow according to claim 1, wherein the plurality of tracks of the algal cells in the step 65 is at least ten thousand or more tracks of the algal cells.
